(19) United States
(12) Patent Application Publication
(10) Pub. No.: US 2004/0215338 A1
(43) Pub. Date: Oct. 28, 2004

(54) Method and system for drug delivery to abdominal aortic or thoracic aortic aneurysms

(76) Inventors: Jeff Elkins, Novato, CA (US); Sandra K. Hathaway, Maastricht (NL); David Tseng, Livermore, CA (US); Michel Letort, Prevessins (FR)

Correspondence Address: FRANK NICHOLAS, Cardinal Law Group, Suite 2000, 1603 Orrington Avenue, Evanston, IL, 60201, US

(21) Appl. No.: 10/422,638

(22) Filed: Apr. 24, 2003

(51) Int. Cl. A61F 2/64

(52) U.S. Cl. 623/1.46

(57) ABSTRACT

The methods and devices for delivering therapeutic agents to abdominal aortic or thoracic aortic aneurysms provide a coated stent graft and methods of using the same. The coated stent graft system 100 comprises a support 20 and graft material 40 attached to the support 20, with a coating 30, 35 including a therapeutic agent disposed on the support 20 and/or the graft material 40. The coated stent graft system 600 can also include a stent graft portion 605 with an agent delivery portion 655 disposed about the stent graft portion 605 and including a therapeutic agent. A stent graft drug delivery system can include an endovascular catheter 710 having a coated region 730 with a therapeutic agent, and a stent graft 705 disposed on the endovascular catheter 710. The endovascular catheter 710 can have a balloon for releasing the therapeutic agent. The therapeutic agent can be injected by endovascular catheter on carrier particles.

### FIELD OF THE INVENTION

This invention relates to medical devices, such as stent grafts. In particular, this invention relates to methods and devices for delivering therapeutic agents to abdominal aortic or thoracic aortic aneurysms.

### BACKGROUND OF THE INVENTION

Abdominal aortic aneurysms (AAA) form in the portion of the aorta that extends through the abdomen. Thoracic aortic aneurysms (TAA) form in the portion of the aorta located within the thoracic cavity. An aneurysm, that is, a bulge or sac that forms in the wall of a blood vessel, causes the vessel wall to lose its elasticity and the force of normal blood pressure in the aneurysm may lead to the rupture of the vessel. Aneurysms are most commonly the result of fatty deposits on the vessel wall but may also result from other causes that weaken the vessel wall, including heredity, trauma or disease.

A number of methods and devices for treating AAA/TAA have been developed. Currently, the standard treatment is conventional open surgery, which is performed to replace the section of the vessel where the aneurysm has formed. The AAA/TAA is accessed by a surgeon through an incision in the abdomen. The portion of the blood vessel where the aneurysm has formed is shut off from the main portion of the blood vessel and then replaced with a synthetic graft. Surgery is performed under general anesthesia and takes three to four hours to complete. Following the surgery, the patient may spend time in an intensive care unit and may remain in the hospital for several days.

For several reasons, including current physical condition of the patient, some patients are not good candidates for such open surgery. Due to the highly invasive nature of the open procedure, some patients may not wish to undergo the treatment. These patients have to live with the continued risk of AAA rupture. Thus, alternative methods of treating an MA or TAA are desirable.

One alternative treatment is a technique known as endovascular stent grafting. In this procedure, a stent graft is placed inside the vessel affected by the aneurysm in order to reinforce the weakened vessel wall, thereby preventing rupture of the aneurysm. The stent graft is guided to the area of the aneurysm using a delivery catheter, typically via the femoral artery and the iliac artery into the aorta. The catheter may be a balloon-expandable catheter and the stent graft may then be deployed and fixed into position by expanding the balloon. Or, in cases of self-expanding stent grafts, retraction of a catheter sheath will deploy the stent graft.

Current stent graft designs for AAA and TAA repair have evolved from simple tubular designs to designs such as Medtronic's AneuRx™ Stent Graft System. The efficacy of endovascular stent grafting is in various stages of clinical evaluation. Follow up studies of 899 patients who underwent AAA stent graft repair between May 1994 and March 1998 showed 90% of the patients were still free from a persistent graft endoleak 18 months post-operation. In some small percentages of patients, however, continued AAA enlargement may occur despite the appearance of exclusion without visible endoleaks. Endoleaks may result in rupture of the aneurysm after treatment. Thus, it would be desirable to provide a stent graft procedure that prevents continued AAA growth.

There has been some evidence that antibiotics, in particular Doxycycline, may be useful in preventing continued AAA growth. Doxycycline has been shown to arrest the aneurysmal process in the aorta by inhibiting the production of the enzymes MMP3 and MMP9 (matrix metalloproteinases 3 and 9 respectively.) These enzymes degrade the structural proteins of the aorta, further weakening the vessel wall. Most therapeutic agents, including doxycycline, increase in efficacy, when closer to their target site.

Additionally, some patients are not eligible for the endovascular stent grafting procedure because their aneurysms are of a prohibitively small diameter. (less than 5 cm). In some cases, these smaller aneurysms are early stage aneurysms. Thus, it would be desirable to provide a stent graft procedure that could treat such smaller aneurysms in an early stage before they progress to a larger, and therefore more dangerous, size.

There has been some evidence that local delivery of certain drugs, such as propranolol and beta-adrenergic blockade, may slow down growth of the aneurysm in the early stages when the diseased tissues are still viable. Moreover, use of certain therapeutic agents, such as anti-inflammatory agents during a surgical procedure may ameliorate the results of the procedure. Additionally, other drugs may be used to slow down dilation of the aneurysm post-operatively, thereby reducing the risk of post-operative rupture. It would be desirable therefore to provide a means for delivering therapeutic agents to endovascular stent graft AAA target sites.

Coated stents have been used effectively in treating atherosclerosis and other forms of coronary narrowing. However, such coated stent procedures address needs specific to atherosclerosis. It would be desirable, therefore, to provide a coated stent graft method and system to address needs specific to abdominal aortic and thoracic aortic aneurysms.

### SUMMARY OF THE INVENTION

One aspect of the present invention provides a coated stent graft system comprising a support and graft material attached to the support, with a coating including a therapeutic agent disposed on the support and/or the graft material. The coating can be dispersed on or impregnated within the support and/or the graft material. The coating can be limited to a portion of the support and/or the graft material, such as the outside of the support and/or the graft material.

Another aspect of the present invention provides a coated stent graft system including a stent graft portion with an agent delivery portion able to be disposed about the stent graft portion and the agent delivery portion including a therapeutic agent.

Another aspect of the present invention provides a stent graft drug delivery system including an endovascular catheter having a coated region with a therapeutic agent, and a stent graft disposed on the endovascular catheter. The, endovascular catheter can have a balloon for releasing the therapeutic agent, or the therapeutic agent can be released by changing physical conditions such as. exposure to blood fluid or temperature. The endovascular catheter can also have a balloon for expanding the stent graft, or the stent graft can be self-expanding.

Another aspect of the present invention provides injecting carrier particles through an endovascular catheter to a target site, before or after a stent graft is deployed at the target site. The present invention further provides a method of and system for manufacturing a coated stent graft.

The foregoing, and other, features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims in equivalence thereof.

### BRIEF DESCRIPTION OF THE DRAWINGS

FIGS. 1 & 2 are a diagrammatic view and a cross section view, respectively, of an AAA/TAA drug delivery system made in accordance with the present invention.

FIG. 3 is a diagrammatic view of another embodiment of a coated stent graft system in accordance with the present invention.

FIG. 4 is a flow chart of a method for delivering therapeutic agents to an AAA/TAA target site in accordance with the present invention.

FIG. 5 is a diagrammatic view of another embodiment of an AAA/TAA coated stent graft system made in accordance with the present invention.

FIGS. 6A & 6B is yet another embodiment of a coated stent graft system in accordance with the present invention.

FIG. 7 is a diagrammatic view of another embodiment of a coated stent graft system according to the present invention.

FIG. 8 is a flow chart of another method for delivering a therapeutic agent to an AAA/TAA target site.

FIG. 9 is a diagrammatic view of another embodiment of an AAA/TAA drug delivery system in accordance with the present invention; and

FIG. 10 is a flow chart of one method for delivering therapeutic agents to an AAA/TAA target site in accordance with the present invention.

FIG. 11 is a process flow chart of one method for manufacturing a coated stent graft system in accordance with the present invention.

### DETAILED DESCRIPTION

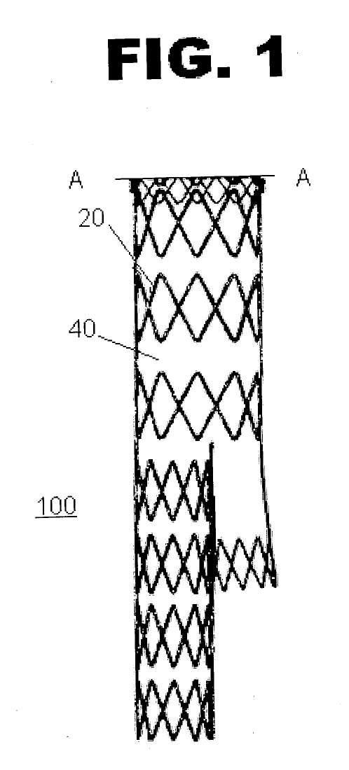

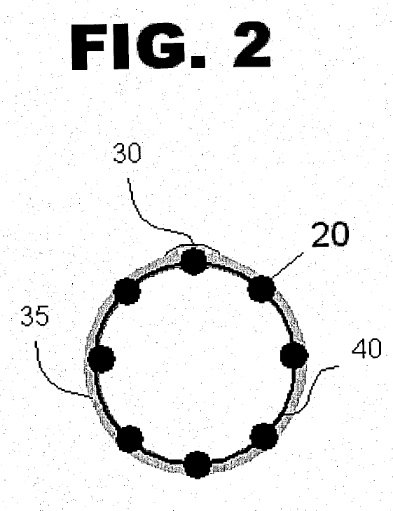

FIGS. 1 & 2 are a diagrammatic view and a cross section view, respectively, of an AAA/TAA drug delivery system made in accordance with the present invention. FIG. 2 shows a cross-section of coated stent graft system 100 taken along A-A of FIG. 1.

FIG. 1 shows a coated stent graft system 100 including a support 20 to which a tubular graft material 40 is attached. A support coating 30 is dispersed on or impregnated within all or a portion of support 20 of coated stent graft system 100. Alternatively, a graft material coating 35 is dispersed on or impregnated within all or a portion of graft material 40 of the coated stent graft system 100. Alternatively, the coatings 30 and 35 are dispersed on or impregnated within all or a portion of support 20. In the embodiment shown in FIGS. 1 & 2, the coatings 30 and 35 are dispersed on the outside of the coated stent graft system 100, i.e., the side facing tissue upon implantation at the target site, thereby coating outside portions of support 20 and of graft material 40.

The coated stent graft system 100 may be any suitable device for mechanically keeping a tubular graft open and in sealing contact with healthy surrounding tissue after being implanted at the target site. Such mechanical endoprosthetic devices, sometimes called stent grafts, are typically inserted into the target vessel, positioned across the lesion, and then expanded to reinforce the weakened wall of the vessel, thereby preventing rupture of the aneurysm while the graft remains in contact with the healthy tissue after implantation of the graft. Generally, the coated stent graft system 100 is placed from just above to just below the aneurysm in a vessel in order to divert flow through the stent graft and relieve the pressure from the weak aneurysm wall.

For example, the coated stent graft system 100 may be a self-expanding and expandable stent graft as is known in the art. Although FIGS. 1 & 2 show a bifurcated stent graft, the coated stent graft system 100 may also be a tubular stent graft, as is known in the art. In one embodiment according to the invention, after the stent graft is positioned across the aneurysm, the stent graft is expanded by the delivery device. Depending on the materials used in construction of the stent graft, the coated stent graft system 100 can maintain the expanded shape through mechanical force, for example.

Support 20 is a support having a suitable mechanical configuration for keeping an effective blood vessel open after completion of the stent grafting procedure. For example, support 20 may be one or more stent type rings attached to graft material 40 and arranged in a manner that will allow coated stent graft system 100 to keep the tubular graft open and in sealing contact with healthy surrounding tissue after implantation. The size and configuration of support 20 depends upon the size and configuration of the vessel to be treated. If stent type rings are used, the number and size of rings used in support 20 depends upon the size and configuration of the vessel to be treated. Individual components, such as individual rings of support 20, may be connected to each other by articulated or rigid joints or may be attached to graft material 40. The minimum length of the coated stent graft system 100 depends on the size of the vessel across which the system 100 will be implanted.

Support 20 is constructed of one or more suitable implantable materials having good mechanical strength. The material can be deformable or self-expandable to produce the deployed shape for the coated stent graft system 100. For example, support 20 may be made of a suitable biocompatible metal, such as implantable quality stainless steel wire. Alternatively, according to the present invention, support 20 is constructed of nitinol or another suitable nickel and titanium alloy. Alternatively, support 20 is constructed of any suitable metallic, plastic or biocompatible material. The outside of the support 20 may be selectively plated with platinum, or other implantable radiopaque substances, to provide improved visibility-during fluoroscopy. The cross-sectional shape of the finished support 20 may be circular, ellipsoidal, rectangular, hexagonal, square, or other polygon, depending on the size and shape of the vessel across which the system is implanted.

Stent graft material 40 is constructed of one or more suitable implantable materials having good tensile strength, such as material suitable for resisting expansion when the force associated with blood pressure is applied to it after completion of the stent grafting procedure. For example, graft material 40 is made of a suitable biocompatible plastic, such as implantable quality woven polyester. In some embodiments, graft material 40 includes components made of collagen, albumin, an absorbable polymer, or biocompatible fiber. Alternatively, graft material 40 is constructed from one or more suitable metallic, plastic, or non-biodegradable materials.

The size and configuration of graft material 40 depends upon the size and configuration of the aneurysm to be treated. For example, the configuration of, graft material 40 is generally tubular as seen in FIG. 1. The size of graft material 40 may be formed to generally match support 20 to which it is attached. According to one embodiment, graft material 40 is formed of one entire woven polyester tube sized to match support 20.

Each coating 30 and 35 may be, for example, a biodegradable coating or a porous non-biodegradable coating, having dispersed therein a sustained-release dosage form of one or more therapeutic agents as described below. In an alternative embodiment, stent graft system 100 also has the therapeutic agent impregnated therein, i.e., within all or a portion of support and/or graft material.

In one embodiment according to the invention, dispersion of the therapeutic agent is nonspecific and the therapeutic agent is released on deployment. In such an embodiment, the therapeutic agent is applied directly to the stent graft system. Because the therapeutic agent is applied to the stent graft system directly, the release of the therapeutic agent in this embodiment will be, immediate upon deployment of the stent graft system 100.

Alternatively, dispersion of the therapeutic agent may be specific. In such an embodiment, the therapeutic agent is applied to the stent graft system in combination with a carrier. The carrier may be adapted to deliver sustained release of therapeutic agent to target cells, e.g., healthy tissue in contact with the stent graft. For example, in accordance with one embodiment of the present invention, an antibiotic such as doxycycline may be delivered to inhibit production of enzymes MMP3 and MMP9 (matrix metalloproteinases 3 and 9, respectively.) These enzymes degrade the structural proteins of the aorta, further weakening the vessel wall. The antibiotic may be combined with a carrier such as a biodegradable polymer. In another example, the antibiotic rifampicin may be used in combination with succinated gelatin as its carrier. In this case the hydroxyl groups of the succinated gelatin binds the rifampicin. Rifampicin is released as the succinated gelatin degrades. Other suitable carriers include, but are not limited to, polylactic acid, polyglycolic acid or collagen.

Thus, the carrier may take the form of a protein, a synthetic polymer, or non-degradable microparticulates or nanoparticulates or biodegradable microparticulates or nanoparticulates. The microparticles or nanoparticles may be formed of a polymer-containing matrix that biodegrades by random, nonenzymatic, hydrolytic scission. One embodiment of the coating is formed of a mixture of thermoplastic polyesters (e.g., polylactide or polyglycolide) or a copolymer of lactide and glypolide components. The lactide/glycolide, structure has the added advantage that biodegradation thereof forms lactic acid and glycolic acid, both normal metabolic products of mammals.

As described above, suitable therapeutic agents include aneurysm dilation prevention therapeutic agents such as propranolol, doxycycline or beta-adrenergic blockade or other therapeutic agents that inhibit MMP activity. Alternatively, the coating may include any suitable therapeutic agent that prevents elastin degradation of the aortic wall. The coating may also include therapeutic agents that slow down growth of the aneurysm in the early stages when the diseased tissues are still viable. Other possible therapeutic agents include anti-inflammatory agents or analgesics that ameliorate the results of the procedure during the procedure or postoperatively. Additionally, the coating may also include therapeutic agents used to slow down dilation of the aneurysm post-operatively, thereby reducing the risk of post-operative rupture. Other therapeutic agents are contemplated including, but not limited to, agents that alter cellular metabolism or are inhibitors of protein synthesis, cellular proliferation, or cell migration; therapeutic agents that affect morphology or increases in cell volume; and/or therapeutic agents that inhibit extracellular matrix synthesis or secretion and cytostatic therapeutic agents that inhibit DNA synthesis and proliferation at doses that have a minimal effect on protein synthesis such as protein kinase inhibitors (e.g., staurosporin), suramin, and nitric oxide releasing compounds (e.g., nitroglycerin) or analogs or functional equivalents thereof. In addition, the coating may also comprise therapeutic agents that inhibit the contraction or migration of smooth muscle cells and maintain an enlarged luminal area following, for example, angioplasty trauma (e.g., the cytochalasins, such as cytochalasin B, cytochalasin C, cytochalasin D or the like).

Other examples of therapeutic agents that may be integrated within the coating include thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti-secretory agents, actin inhibitors, remodeling inhibitors, antisense nucleotides, anti-metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, inhibitors, free radical scavengers, chelators, antioxidants, antipolymerases, antiviral agents, photodynamic therapy agents, and gene therapy agents. The coating may also be a conjugate of several therapeutic substances.

The dosage of therapeutic agents included in the coating is varied depending on the body lumen involved, the result desired, and the therapy indicated. Preferably therapeutic agents are dispersed within the microparticulates or nanoparticulates of the coating as described above. The dosage forms of the coating may be targeted to a relevant target cell population by a binding protein or peptide. For example, to target the cells in contact with an endovascular stent graft, a coating of collagen may be used.

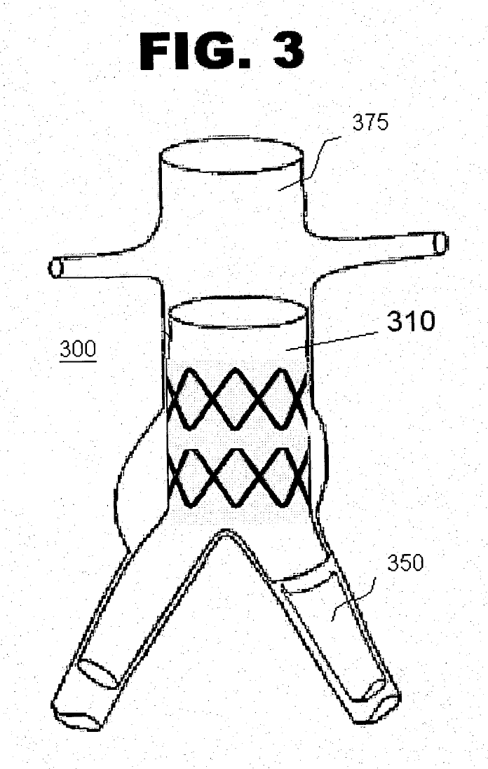

FIG. 3 is a diagrammatic view of another embodiment of a coated stent graft system in accordance with the present invention. In this embodiment, the coated stent graft system 300 is made of at least two stent grafts that combine to form the coated stent graft system 300. The coated stent graft system 300 comprises a first stent graft portion 310 and a second stent graft portion 350, shown in place within vessel 375. The first stent graft portion 310 and second stent graft portion 350 may each comprise a support and graft material covering the support. Both first stent graft portion 310 and second stent graft portion 350 may be coated according to the present invention. Alternatively, only first stent graft portion 310 or second portion 350 may be coated. The second stent graft portion 350 may be installed so that part of the second stent graft portion 350 is disposed within the first stent graft portion 310, so that the first stent graft portion 310 and second stent graft portion 350 form a continuous unit. The first stent graft portion 310 and second stent graft portion 350 may be positioned and expanded using a balloon catheter.

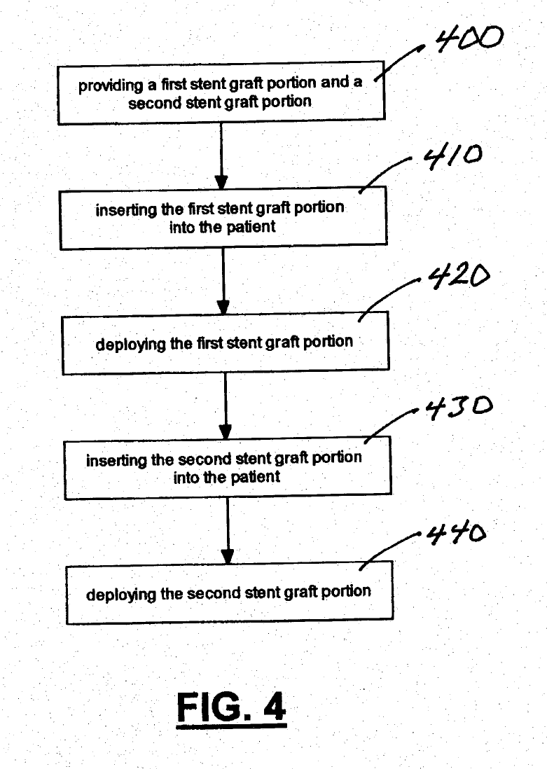

FIG. 4 is a flow chart of a method for delivering therapeutic agents to an AAA/TAA target site in accordance with the present invention. A first stent graft portion and a second stent graft portion are provided at 400. The first stent graft portion is inserted into the patient at 410 and deployed at 420. The second stent graft portion is inserted into the patient at 430 and deployed at 440. Those skilled in the art will appreciate that the order of steps presented is exemplary only and may be varied to suit particular requirements.

At 400, a first stent graft portion and second stent graft portion are provided. The first and second stent graft portions may each comprise a support and graft material covering the support. Both first and second stent graft portions may contain a therapeutic agent or only the first or second stent graft portion may contain a therapeutic agent. The first stent graft portion may be a bifurcated stent graft and the second stent graft portion may be a leg stent graft adapted to fit with the first stent-graft portion.

The first stent graft portion is inserted into the patient at 410. The first stent graft portion may be guided to the location of the target AAA/TAA aneurysm using a delivery catheter, typically via the femoral artery and the iliac artery into the aorta. The first stent graft portion is deployed at 420. When the catheter is in place at the aneurysm, the first stent graft portion carried by the catheter may be deployed by inflating a balloon portion of the catheter. The balloon portion may then be deflated and the catheter removed.

The second stent graft portion is inserted into the patient at 430. A second small incision in the patient's left thigh may then be made and thee catheter may be threaded up to the first stent graft portion previously installed. The second stent graft portion or “leg” of the coated stent graft system is carried on the catheter. The second stent graft portion is deployed at 440. When the catheter is in the desired position at the first stent graft portion, the second stent graft portion is deployed by inflating a balloon portion of the catheter. The balloon portion may then be deflated and the catheter removed.

After implantation of the stent graft system, the therapeutic agent is released from the polymer carriers at the target site over time. For example, if the graft material is an absorbable material that has been dip-coated, therapeutic agents in the coating may then “weep” out of the absorbable material at the target surgical site—bathing the sac, thrombus, and blood tissue in the therapeutic agent. Alternatively, if the stent graft is spray coated using a suitable polymer/solvent mixture, the therapeutic agent will be released at a steady rate overtime.

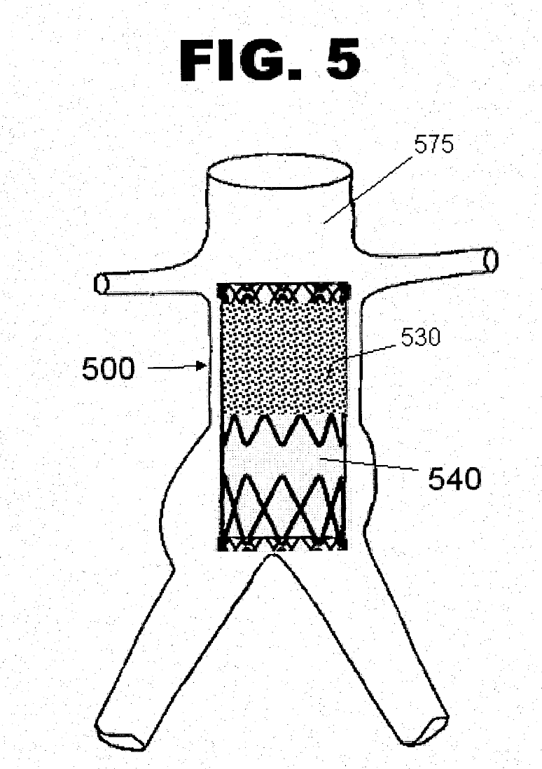

FIG. 5 is a diagrammatic view of another embodiment of an AAA/TAA coated stent graft system made in accordance with the present invention. In this embodiment, the coated stent graft system 500 has a stent coated portion 530 and a stent uncoated portion 540. A tubular stent graft is shown deployed in vessel 575 as an example, although the advantages apply equally to a bifurcated stent graft. The stent coated portion 530 is contains or coated with a therapeutic agent and the stent uncoated portion 540 does not include a therapeutic agent. Those skilled in the art will appreciate that the coated stent graft system may have a number of coated and uncoated regions in different patterns as required for a particular therapy. Further, the different stent coated portions on one coated stent graft system may contain different therapeutic agents.

In one example, the stent coated portion 530 may be located on the coated stent graft system 500 in the area shown in FIG. 5 in the critical area of the proximal seal between the coated stent graft system 500 and the vessel 575. Studies indicate that the proximal necks of the vessel 575 dilate in this area following implant of a conventional graft stent. Local delivery of a suitable therapeutic agent, such as doxycycline, to this critical area from a stent coated portion may arrest such dilation. In another example, areas of the coated stent graft system may be left uncoated in areas where a coating may cause excessive friction or other adverse reactions with the vessel.

The stent coated portion 530 may be coated by spraying the therapeutic agent onto the support and graft material. Particular patterns may be obtained by spraying the therapeutic agent through a mask. Those skilled in the art will appreciate that the therapeutic agent may be applied by a number of methods, including rolling, dipping, spraying, printing, and ink jet printing.

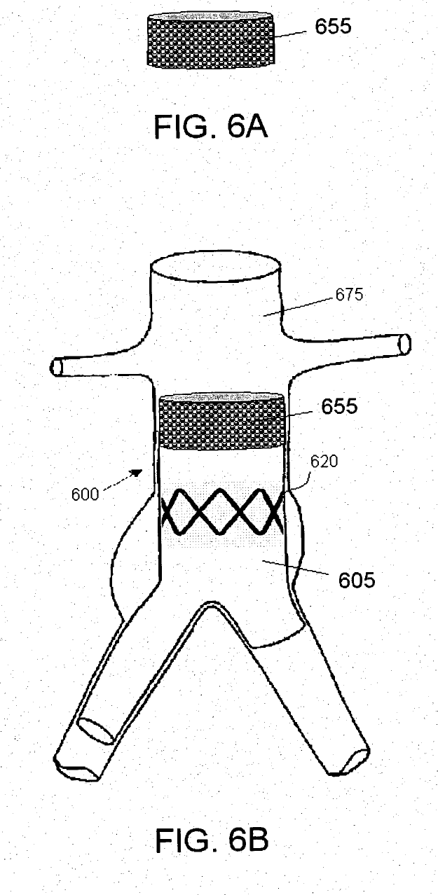

FIGS. 6A & 6B, in which like elements share like reference numbers, is yet another embodiment of a coated stent graft system in accordance with the present invention. FIG. 6A shows an agent delivery portion 655 alone, while FIG. 6B shows the agent delivery portion 655 disposed on a stent graft portion 605. The coated stent graft system 600 comprises a stent graft portion 605 and an agent delivery portion 655 disposed about the stent graft portion 605. The stent graft portion 605 may be a bifurcated stent graft as illustrated, or a tubular stent graft. The agent delivery portion 655 may be a ring or cuff to be placed between the stent graft portion 605 and the vessel 675. The agent delivery portion 655 is coated and the stent graft portion 605 remains uncoated. It may be desirable to leave the stent graft itself uncoated if the coating causes excessive friction within vessel 675. In other embodiments, the stent graft portion 605 may be coated as well.

Agent delivery portion 655 is attached to stent graft portion 605 in combination with support 620 and provides additional mechanical structure to the stent graft portion 605. Agent delivery portion 655 is constructed of one or more suitable implantable materials having good tensile strength, such as a material suitable for resisting expansion when the force associated with blood pressure is applied after completion of the stent grafting procedure. The material of agent delivery portion 655 is preferably chosen so that agent delivery portion 655 is highly absorbable and will “weep out” the therapeutic agent. Thus, agent delivery portion 655 may be made of a suitable biocompatible plastic, such as implantable quality woven polyester and may include components made of collagen, of albumin, of an absorbable polymeror of biocompatible fiber.

The agent delivery portion 655 may also comprise one or more suitable metallic, plastic or non-biodegradable material providing additional strength to stent graft portion 605. The size and configuration of agent delivery portion 655 depends upon the area of the vessel where portion 655 will be placed. For example, the configuration of agent delivery portion 655 may be tubular to fit within the proximal seal area.

The agent delivery portion 655 is dip coated or spray coated with the therapeutic agent, allowed to dry, and attached to the graft material of stent graft portion 605. The coating may be applied to agent delivery portion 655 by dipping or spraying the agent delivery portion using the method described above. Agent delivery portion 655 is then placed in any suitable area along stent graft portion 605, including, for example, the critical area at the proximal seal between the stent graft 605 and vessel 675.

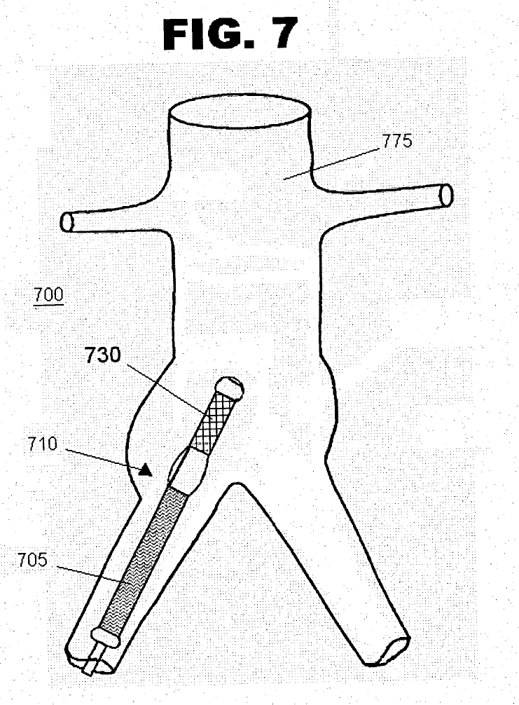

FIG. 7 is a diagrammatic view of another embodiment of a coated stent graft system according to the present invention. A catheter is used to deliver a desired therapeutic agent locally during stent graft implantation. The coated stent graft system 700 comprises an endovascular catheter 710 having at least one coated region 730 and a stent graft 705 disposed on the endovascular catheter 710. The coated region 730 further includes one or more suitable therapeutic agents dispersed therein. The stent graft 705 may be a balloon expandable or self-expanding coated stent graft, such as a bifurcated stent graft or a tubular stent graft. The endovascular catheter 710 delivers the therapeutic agents into vessel 775 and expands the stent graft 705.

The catheter 710 is an endovascular catheter as is well known in the art. Any conventional or modified balloon catheter may also be used. For example, the catheter 710 may be a low profile design with a tapered distal tip, and an inner lumen for insertion of a conventional guide wire. The catheter 710 generally includes an expandable balloon portion. This expandable portion is formed from a material such as polyethylene, polyethylene terephthalate (PET), nylon, or the like. The length and diameter of the balloon is selected to accommodate the particular configuration of the catheter 705. In one embodiment, stent graft 705 is disposed on the expandable balloon portion and catheter 710 is used to deploy stent graft 705 to the target site.

The coated region 730 comprises a matrix adapted to deliver a therapeutic agent to target cells. The matrix may be a biodegradable polymer, such as PLA, PLGA, or collagen. In one embodiment, the coated region 730 is formed of a mixture of thermoplastic polyesters e.g., polylactide or polyglycolide, or, a copolymer of lactide and glycolide components. The lactide/glycolide structure has the added advantage that biodegradation thereof forms lactic acid and glycolic acid, both normal metabolic products of mammals. Other suitable matrix components include, but are not limited to, succinated gelatin, proteins, and synthetic polymers. The coating in the coated region 730 may be applied by a number of methods known to those skilled in the art, including rolling, dipping, spraying, printing, and ink jet printing.

The coated region 730 may also take the form of non-degradable microparticulates or nanoparticulates or biodegradable microparticulates or nanoparticulates. The microparticles or nanoparticles may be formed of a polymer-containing matrix that biodegrades by random, nonenzymatic, hydrolytic scission.

The coated region 730 may further include, disposed therein, one or more suitable therapeutic agents for delivering therapy to the target site. Examples of suitable therapeutic agents include, but are not limited to, aneurysm dilation prevention therapeutic agents such as propranolol, doxycycline and beta-adrenergic blockade; MMP activity inhibitors; agents that prevent elastin degradation of the aortic wall; analgesics and anti-inflammatory agents; and antibiotics such as rifampicin.

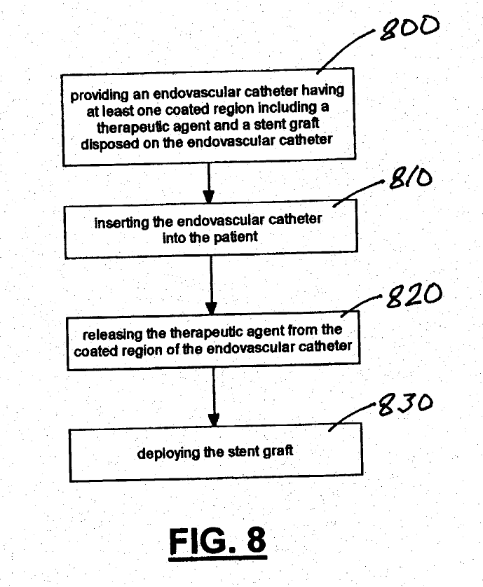

FIG. 8 is a flow chart illustrating a method for delivering therapeutic agents to an AAA/TAA target site using the embodiment of FIG. 7. An endovascular catheter having at least one coated region including a therapeutic agent and a stent graft disposed on the endovascular catheter is provided at 800. The endovascular catheter is inserted into the patient at 810. At 820, the therapeutic agent is released from the coated region of the endovascular catheter. The stent graft is deployed at 830. Those skilled in the art will appreciate that the order of steps presented is exemplary only and may be varied to suit particular requirements.

At 800, an endovascular catheter having at least one coated region including a therapeutic agent and a stent graft disposed on the endovascular catheter is provided. The coated region comprises a matrix adapted to deliver a therapeutic agent to target cells. The stent graft may be a balloon expandable, coated stent graft, such as a bifurcated stent graft or a tubular stent graft. In other embodiments, the stent graft may be self-expanding.

At 810, the endovascular catheter is inserted into the patient with the distal end at the location of the target AAA/TAA aneurysm. Typically, the endovascular catheter is inserted via the femoral artery and the iliac artery into the aorta.

At 820, the therapeutic agent is released from the coated region of the endovascular catheter at the target location. The therapeutic agent may be released by the change in physical conditions, such as the exposure to blood fluid or temperature. In another embodiment, the therapeutic agent may be released by physical action, such as inflating a balloon region below the coated region to dislodge the therapeutic agent from the coated region.

The stent graft is deployed at the target location at 830. When the endovascular catheter is in place at the aneurysm, the first stent graft portion carried by the endovascular catheter may be deployed by inflating a balloon portion of the endovascular catheter. The balloon portion may then be deflated and the endovascular catheter removed.

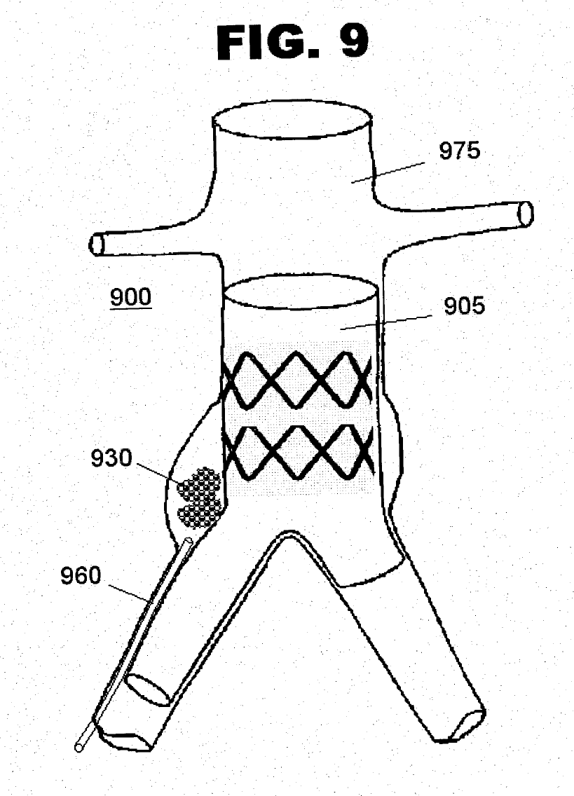

FIG. 9 is a diagrammatic view of another embodiment of an AAA/TAA drug delivery system in accordance with the present invention; and the coated stent graft system 900 comprises a stent graft 905 with carrier particles 930 disposed in an aneurysm between the stent graft 905 and vessel 975. The carrier particles 930 are suitable particles for carrying therapeutic agents, such as microspheres of polyvinyl alcohol (PVA) coated with a therapeutic agent. An endovascular catheter or needle 960 is used to deliver the carrier particles 930 to the target site. The stent graft 905 may be a tubular or bifurcated coated stent graft. The catheter 960 may be any catheter that may be inserted in an aneurysm between the stent graft 905 and vessel 975.

The carrier particles 930 may comprise polymer particles able to deliver sustained release of therapeutic agent to target cells. Such a matrix may be a biodegradable polymer, such as PLA (polylactic acid), PLGA (poly lactic-co-glycolic acid), or collagen. Such carrier particles, particularly PVA microspheres, are well characterized in the art. The carrier particles 930 may also take the form of non-degradable microparticulates or nanoparticulates or biodegradable microparticulates or nanoparticulates. The microparticles or nanoparticles may be formed of a polymer-containing matrix that biodegrades by random, nonenzymatic, hydrolytic scission.

In one embodiment, the carrier particles 930 are PVA particles that have been dipped, or otherwise coated or impregnated, with a therapeutic agent. As is characterized in the art, microspheres, particularly PVA microspheres, have a high density and are capable of absorbing other material, such as suspensions of therapeutic agents. The PVA microspheres are highly expandable and capable of holding large volumes of liquid.

Examples of suitable therapeutic agents that may be carried in the carrier particles 930 include, but are not limited to, aneurysm dilation prevention therapeutic agents such as propranolol, doxycycline and beta-adrenergic blockade; MMP activity inhibitors; agents that prevent elastin degradation of the aortic wall; analgesics and anti-inflammatory agents.

The microspheres are impregnated or coated with the desired therapeutic agent. One process involves suspending carrier particles in a suitable solvent, including but are not limited to chloroform, THF (tetrahydrofuran), and DMSO (dimethyl sulfoxide). The desired therapeutic agent may then be added to the solvent/polymer solution. The carrier particles absorb the therapeutic agent. This solvent/carrier particles/therapeutic agent combination may then be injected or infused at the target site.

In one exemplary embodiment, polylactide polymer may be dissolved in chloroform solvent. A doxycycline therapeutic agent may then be added to the solvent/polymer solution. PVA microsphere carriers are added to absorb the solvent/polymer/therapeutic agent solution for injection. For example, doxycycline as the therapeutic agent is added to a polylactide/chloroform mixture to provide a 30-50 percent concentration of therapeutic agent to solvent/polymer. A suitable amount of doxycycline for providing a two-year release of the drug is two grams, which provides a release rate of about two mg/day at the target site. If a shorter release period is desired, less doxycycline may be used.

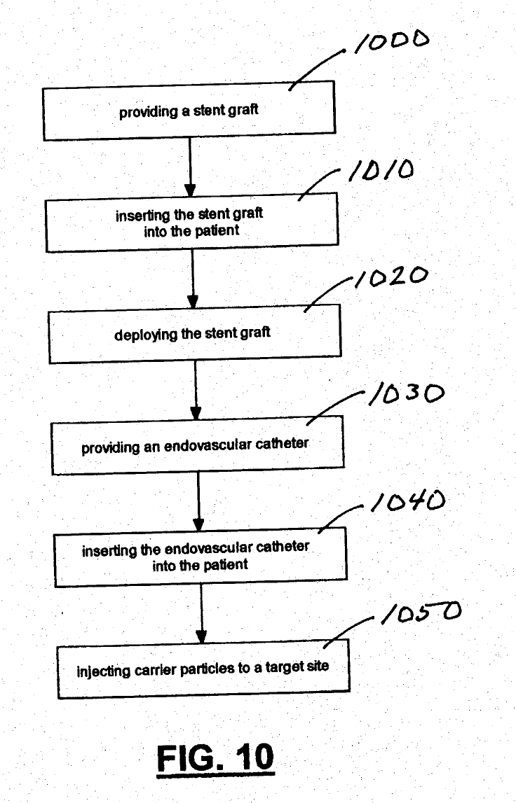

FIG. 10 is a flow chart of one method for delivering therapeutic agents to an AAA/TAA target site in accordance with the present invention. A stent graft is provided at 1000. The stent graft is inserted into the patient at 1010 and deployed at 1020. An endovascular catheter is provided at 1030 and inserted into the patient at 1040. The distal end of the endovascular catheter is disposed at the target site in the aneurysm between the stent graft and vessel. Carrier particles are injected to the target site at 1050. Those skilled in the art will appreciate that the order of steps presented is exemplary only and may be varied to suit particular requirements.

The therapeutic agent may be injected during or after the stent graft procedure for a therapeutic effect during or after the stent graft implantation. For example, an antibiotic or anti-dilation effect may be beneficial during or after stent graft implantation. Carrier particles may also be left in the aneurysm sac for long-term therapeutic effect post-implantation. In another embodiment, the carrier particles can be injected to the target site long after the initial stent graft implantation.

At 1000, a stent graft is provided. The stent graft may comprise a support and graft material covering the support. Both support and graft material may contain a therapeutic agent, or only one or the other may contain a therapeutic agent. The stent graft may be a tubular or a bifurcated stent graft.

The stent graft is inserted into the patient at 1010. The stent graft may be guided to the location of the target AAA/TAA aneurysm using a delivery catheter, typically via the femoral artery and the iliac artery into the aorta. The stent graft is deployed at 1020. When the delivery catheter is:in place at the aneurysm, the stent graft carried by the catheter may be deployed by inflating a balloon portion of the delivery catheter. The balloon portion may then be deflated and the delivery catheter removed.

An endovascular catheter is provided at 1030. The endovascular catheter may be any catheter suitable for maneuvering into the aneurysm between the stent graft and vessel, and capable of delivering the carrier particles. The endovascular catheter is inserted into the patient at 1040 with the distal end of the endovascular catheter disposed at the target site in the aneurysm between the stent graft and vessel. The carrier particles are injected to the target site at 1050 and the endovascular catheter may be removed.

In another method of delivering therapeutic agents to an AAA/TAA target site, the carrier particles may be delivered to the target site before the stent graft is deployed. The carrier particles with therapeutic agent are injected before the stent graft is placed and the aorta and aneurysm sac are bathed in the therapeutic agent before the stent graft procedure takes place. This may be suitable when a therapeutic effect is desired pre-implantation, such as for an analgesic or anti-inflammatory effect. If the carrier particles release therapeutic agent over time, pre-implantation may also be useful for providing long-term therapeutic effect, such as anti-dilation.

Initially, the blood vessel upstream of the aneurysm may be occluded with a balloon catheter or other methods of occluding a blood vessel well known in the art. The microsphere carriers are then delivered to the target site by injection or infusion catheter. In one embodiment, the carriers are delivered to the target site using a catheter needle which may be part of the guide catheter used to direct the stent graft into position. The carriers may be delivered directly into the aneurysmal sac so that the carriers release the therapeutic agent over time directly to the aneurismal tissue. For example, the PVA particles describe above may continuously release the therapeutic agent into the aneurismal sac post-implantation.

Finally, a stent graft is deployed to the target site. In one embodiment, the stent graft is disposed on the catheter used to inject the carrier particles to the target site. Thus, the carrier particles are injected or infused locally before the stent graft implantation. In some embodiments, the stent graft is also coated. This allows delivery of a therapeutic agent from the carrier particles and delivery of the same or different agent from the stent graft.

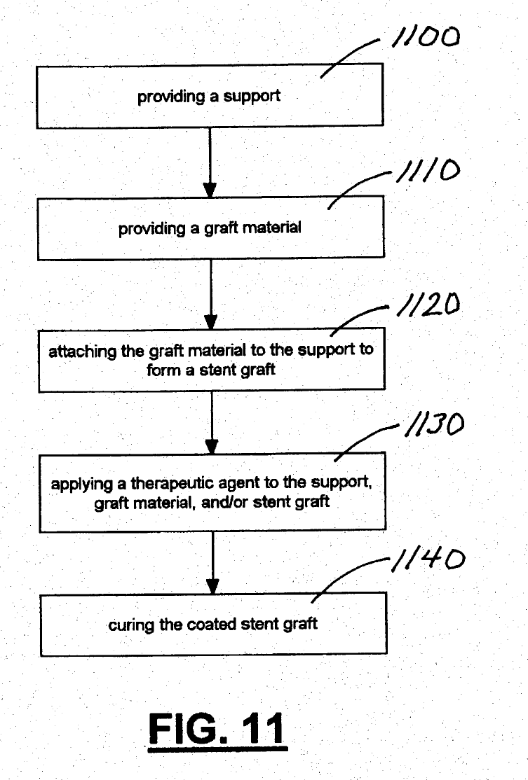

FIG. 11 is a process flow chart of one method of manufacturing a coated stent graft system. A support is provided at 1100 and graft material is provided at 1110. The graft material is attached to the support at 1120 to form a stent graft. A therapeutic agent is applied to the support, graft material, and/or stent graft at 1130. The coated stent graft is then dried or cured at 1140. Those skilled in the art will appreciate that the order of manufacturing steps presented is exemplary only and may be varied to suit particular manufacturing requirements.

At 1100, a support is provided. The support may be uncoated or coated with a therapeutic agent. If coated, the therapeutic agent may be disposed uniformly over the support or may be on portions of the support. Those skilled in the art will appreciate that a solvent/polymer solution containing the therapeutic agent may be applied to the support by a number of methods, including rolling, dipping, spraying, printing, and ink jet printing.

The support is constructed of one or more suitable implantable materials having good mechanical-strength. For example, the support may be made of stainless steel, nitinol, nickel-titanium alloy, or any other suitable metallic, plastic or biocompatible material. The outside of the support may be selectively plated with platinum, or other implantable radiopaque substances, to provide improved visibility during fluoroscopy. The cross-sectional shape of the support in the expanded condition may be circular, ellipsoidal, rectangular, hexagonal, square, or other-polygon, depending on the size and shape of the vessel across which the system is implanted.

A graft material is provided at 1110. The graft material may be impregnated or coated with a therapeutic agent, or be without a therapeutic agent. The therapeutic agent may be disposed uniformly over a sheet of graft material or may be applied in a pattern such that only a portion of the aneurysm will be exposed to the therapeutic agent when the graft stent is deployed at the target site. Those skilled in the art will appreciate that a solvent/polymer solution containing the therapeutic agent may be applied to the graft material by a number of methods, including rolling, dipping, spraying, printing, and ink jet printing.

The graft material is constructed of one or more suitable implantable materials having good tensile strength, suitable for resisting expansion from blood pressure after deployment of the stent graft. For example, the graft material may be made of biocompatible plastic; woven polyester; material including collagen, albumen, absorbable polymer, or biocompatible fiber; or any other suitable metallic, plastic, or non-biodegradable material.

The stent graft material may be dipped, sewn closed, and then attached to the support. For example, the graft material may be shaped into a desired configuration prior to dipping and is then allowed to dry in the desired shape. The support is attached to the graft material after the graft material has dried. If the graft material is dipped before having the support attached, the graft material should be of a suitable density so that the material does not become brittle after dipping. For example, one suitable material is Type 56T DuPont Dacron® polyester fibers.

The graft material is attached to the support at 1120 to form a stent graft. The graft material may be attached by sewing or with rings of a biocompatible fiber or metal, such as stainless steel or nitinol. In other embodiments, the graft material may be attached by gluing with an adhesive, such as acrylic glue. The graft material is typically attached to the inside of the support, although in other embodiments the graft material may be attached to the outside of the support.

At 1130, a therapeutic agent is applied to the support, graft material, and/or stent graft. The therapeutic agent can be applied to the support or graft material before they are combined to form the stent graft. The therapeutic agent may be applied to the whole stent graft or to portions of the stent graft, e.g., the therapeutic agent may be applied in rings around the outside of the stent graft, or may be applied to the inside and not the outside of the stent graft. This step is optional if the support or graft material already contains a therapeutic agent. In another embodiment, the application at 1130 may be used to apply new or different therapeutic agents, than those already contained in the support or graft material.

In one embodiment, the stent graft may be dip coated with a suitable polymeric carrier containing a desired therapeutic agent. For example, the resin of the polymeric carrier may be dissolved in a suitable solvent including, but not limited to, chloroform, tetrahydrofuran (THF), or dimethyl sulfoxide (DMSO). The desired therapeutic agent is added to the solvent/polymer solution. The stent graft is then dipped into the solvent/polymer solution containing the therapeutic agent. In one embodiment of the invention, the entire stent graft is dipped together so that the outside and the inside of the stent graft are coated.

In another embodiment, the stent graft may be spray coated with a suitable polymeric carrier containing a desired therapeutic agent, such as by using ultrasound spray coating. The resin of the polymeric carrier may be dissolved in a suitable solvent including, but not limited to, chloroform, THF, or DMSO. The desired therapeutic agent is added to the solvent/polymer solution. In one embodiment, doxycycline is used as the therapeutic agent and is added to the solvent/polymer mixture to provide a 30 to 50 percent concentration of therapeutic agent to solvent/polymer. The solvent/polymer solution containing the therapeutic agent may then be pumped through an ultrasound spray nozzle onto the stent graft. Ultrasound spray nozzles provide the ability to control the pressure at which and the time for which the solution is sprayed. The solution forms a mist on the surface of the graft material, which accumulates on the surface as the coating. The spraying duration determines the coating thickness. In one embodiment, the stent graft is coated to a thickness of 50 microns.

Those skilled in the art will appreciate that the solvent/polymer solution containing the therapeutic agent may be applied to the stent graft by a number of methods, including rolling, dipping, spraying, printing, and ink jet printing.

The coated stent graft is dried or cured at 1140 as required for the particular solvent/polymer solution containing the therapeutic agent. The stent graft may be air dried or dried in a controlled atmosphere.

It will be appreciated by those skilled in the art that while specific embodiments according to the present invention have been described above, numerous other uses, modifications and departures fall within the spirit and scope of the description.

We claim:

1. A stent graft system, comprising:.
a support 20;
a graft material 40 attached to the support 20; and
a graft material coating 35 disposed on the graft material 40, the graft material coating 35 including a therapeutic agent.

2. The stent graft system of claim 1 wherein the graft material coating 35 is disposed on the graft material 40 in a manner selected from the group consisting of being dispersed on the graft material 40 and being impregnated within the graft material 40.

3. The stent graft system of claim 1 wherein the therapeutic agent is selected from the group consisting of an antibiotics, doxycycline, rifampicin, propranolol, beta-adrenergic blockades, MMP activity inhibitors, elastin degradation preventatives, anti-inflammatory agents, analgesics, and anti-dilation agents.

4. The stent graft system of claim 1 wherein the support 20 is constructed from at least one material selected from the group consisting of biocompatible metal, implantable quality stainless steel wire, nitinol, nickel and titanium alloy, and biocompatible plastic.

5. The stent graft system of claim 1 wherein the graft material 40 is constructed from at least one material selected from the group consisting of biocompatible plastic, implantable quality woven polyester, collagen, albumin, absorbable polymer, biocompatible fiber, and biocompatible metal.

6. The stent graft system of claim 1 wherein the graft material 40 has an inside and an outside, the graft material coating 35 being dispersed on the outside of the graft material 40 and the inside of the graft material 40 being uncoated.

7. The stent graft system of claim 1 wherein the graft material coating 35 includes a carrier.

8. The stent graft system of claim 7 wherein the carrier comprises at least one component selected from the group consisting of biodegradable polymers, succinated gelatin, polylactic acid, polyglycolic acid, collagen, proteins, synthetic polymers, non-degradable microparticulates, non-degradable nanoparticulates, biodegradable microparticulates, biodegradable nanoparticulates, thermoplastic polyesters, and copolymers of lactide and glycolide.

9. The stent graft system of claim 1 wherein the graft material 40 has a first portion and a second portion, the graft material coating 35 being disposed on the first portion and the second portion being uncoated.

10. A stent graft system, comprising:
a support 20;
a graft material 40 attached to the support 20; and
a support coating 30 disposed on the support 20, the support coating 30 including a therapeutic agent.

11. The stent graft system of claim 10 wherein the support coating 30 is disposed on the support 20 in a manner selected from the group consisting of being dispersed on the support 20 and being impregnated within the support 20.

12. The stent graft system of claim 10 wherein the therapeutic agent is selected from the group consisting of antibiotics, doxycycline, rifampicin, propranolol, beta-adrenergic blockades, MMP activity inhibitors, elastin degradation preventatives, anti-inflammatory agents, analgesics, and anti-dilation agents.

13. The stent graft system of claim 10 wherein the support 20 is constructed from at least one material selected from the group consisting of biocompatible metal, implantable quality stainless steel wire, nitinol, nickel and titanium alloy, and biocompatible plastic.

14. The stent graft system of claim 10 wherein the graft material 40 is constructed from at least one material selected from the group consisting of biocompatible plastic, implantable quality woven polyester, collagen, albumin, absorbable polymer, biocompatible fiber, and biocompatible metal.

15. The stent graft system of claim 10 wherein the support 20 has an inside and an outside, the support coating 30 being dispersed on the outside of the support 20 and the inside of the support 20 being uncoated.

16. The stent graft system of claim 10 wherein the support coating 30 includes a carrier.

17. The stent graft system of claim 16 wherein the carrier comprises at least one component selected from the group consisting of biodegradable polymers, succinated gelatin, polylactic acid, polyglycolic acid, collagen, proteins, synthetic polymers, non-degradable microparticulates, non-degradable nanoparticulates, biodegradable microparticulates, biodegradable nanoparticulates, thermoplastic polyesters, and copolymers of lactide and glycolide.

18. The stent graft system of claim 10 wherein the support 20 has a first portion and a second portion, the support coating 30 being disposed on the first portion and the second portion being uncoated.

19. A coated stent graft system comprising:
a stent graft portion 605; and
an agent delivery portion 655, the agent delivery portion 655 able to be disposed about the stent graft portion 605 and including a therapeutic agent.

20. The coated stent graft system of claim 19 wherein the therapeutic agent is selected from the group consisting of an antibiotics, doxycycline, rifampicin, propranolol, beta-adrsnergic blockades, MMP activity inhibitors, elastin degradation preventatives, anti-inflammatory agents, analgesics, and anti-dilation agents.

21. The coated stent graft system of claim 19 wherein the agent delivery portion 655 is constructed from at least one material selected from the group consisting of biodegradable polymers, succinated gelatin, polylactic acid, polyglycolic acid, collagen, proteins, synthetic polymers, non-degradable microparticulates, non-degradable nanoparticulates, biodegradable microparticulates, biodegradable nanoparticulates, thermoplastic polyesters, and copolymers of lactide and glycolide.

22. A stent graft drug delivery system comprising:
an endovascular catheter 710, the endovascular catheter 710 having a coated region 730, the coated region 730 including a therapeutic agent; and
a stent graft 705 disposed on the endovascular catheter 710.

23. The stent graft drug delivery system of claim 22 wherein the stent graft 705 is a coated stent graft.

24. The stent graft drug delivery system of claim 22 wherein the therapeutic agent is selected from the group consisting of antibiotics, doxycycline, rifampicin, propranolol, beta-adrenergic blockades, MMP activity inhibitors, elastin degradation preventatives, anti-inflammatory agents, analgesics, and anti-dilation agents.

25. The stent graft drug delivery system of claim 22 wherein the graft material coating 35 includes at least one component selected from the group consisting of biodegradable polymers, succinated gelatin, polylactic acid, polyglycolic acid, collagen, proteins, synthetic polymers, non-degradable microparticulates, non-degradable nanoparticulates, biodegradable microparticulates, biodegradable nanoparticulates, thermoplastic polyesters, and copolymers of lactidel and glycolide.

26. The stent graft drug delivery system of claim 22 wherein the therapeutic agent is released by an event selected from the group consisting of a change in physical conditions and a physical action.

27. The stent graft drug delivery system of claim 22 wherein the endovascular catheter 710 has at least one balloon portion.

28. The stent graft drug delivery system of claim 27 wherein the coated region 730 is disposed on the balloon portion.

29. The stent graft drug delivery system of claim 27 wherein the stent graft 705 is disposed on the balloon portion.

30. The stent graft drug delivery system of claim 22 wherein the stent graft 705 is self-expanding.

31. A method for delivering a stent graft comprising:
providing an endovascular catheter having at least one coated region including a therapeutic agent, and a stent graft disposed on the endovascular catheter 800;
inserting the endovascular catheter into the patient 810;
releasing the therapeutic agent from the coated region 820; and
deploying the stent graft 830.

32. The method of claim 31 wherein the stent graft is a coated stent graft.

33. The method of claim 31 wherein the endovascular catheter has a balloon, the coated region is disposed on the balloon, and releasing the therapeutic agent from the coated region comprises inflating the balloon.

34. The method of claim 31 wherein the endovascular catheter has a balloon, the stent graft is disposed on the balloon, and deploying the stent graft comprises inflating the balloon.

35. A method for stent graft drug delivery at a target site comprising:
providing a stent graft 1000;
inserting the stent graft into the patient at the target site 1010;
deploying the stent graft 1020;
providing an endovascular catheter 1030;
inserting the endovascular catheter into the patient to the target site 1040; and
injecting carrier particles through the endovascular catheter to the target site 1050.

36. The method of claim 35 wherein the stent graft is a coated stent graft.

37. The method of claim 35 wherein injecting carrier particles through the endovascular catheter to the target site comprises injecting carrier particles through the endovascular catheter into an aneurysm.

38. The method of claim 35 wherein the carrier particles are made of a material selected from the group consisting of biodegradable polymers, polyvinyl alcohol, succinated gelatin, polylactic acid, polyglycolic acid, collagen, proteins, synthetic polymers, non-degradable microparticulates, non-degradable nanoparticulates, biodegradable microparticulates, biodegradable nanoparticulates, thermoplastic polyesters, and copolymers of lactide and glycolide.

39. The method of claim 35 wherein the carrier particles include a therapeutic agent selected from the group consisting of antibiotics, doxycycline, rifampicin, propranolol, beta-adrenergic blockades, MMP activity inhibitors, elastin degradation preventatives, anti-inflammatory agents, analgesics, and anti-dilation agents.

40. The method of claim 35 wherein providing a stent graft comprises providing a stent graft disposed on a balloon of a delivery catheter, and deploying the stent graft comprises inflating the balloon.

41. The method of claim 35 further comprising occluding a blood vessel upstream of the target site.

42. A method of manufacturing a coated stent graft comprising:
providing a support 1100;
providing graft material 1110;
attaching the graft material to the support to form a stent graft 1120; and
applying a therapeutic agent to at least one element selected from the group consisting of the support, the graft material, and the stent graft 1130.

43. The method of claim 42 further comprising curing the coated stent graft 1140.

44. The method of claim 42 wherein attaching the graft material to the support comprises attaching the graft material to the support by a method selected from the group consisting of sewing and gluing.

45. The method of claim 42 wherein the therapeutic agent is contained in a solvent/polymer solution.

46. The method of claim 42 wherein applying a therapeutic agent to at least one element comprises uniformly coating the element.

47. The method of claim 42 wherein applying a therapeutic agent to at least one element comprises coating a portion of the element.

48. The method of claim 42 wherein applying a therapeutic agent to at least one element comprises coating the element by a method selected from the group comprising rolling, dipping, spraying, printing, and ink jet printing.

49. A system for manufacturing a coated stent graft comprising:
means for attaching a graft material to a support to form a stent graft; and
means for applying a therapeutic agent to at least one element selected from the group consisting of the support, the graft material, and the stent graft.

50. The system of claim 49 further comprising means for curing the coated stent graft.
